(19) United States
(12) Patent Application Publication
(10) Pub. No.: US 2008/0208305 A1
(43) Pub. Date: Aug. 28, 2008

(54) APPARATUS AND METHODS FOR TREATING PULMONARY CONDITIONS

(75) Inventors: Ali R. Rezai, Shaker Heights, OH (US); Medhi Ansarinia, Las Vegas, NV (US); Milind Deogaonkar, Broadview Heights, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 12/016,115

(22) Filed: Jan. 17, 2008

Related U.S. Application Data
(60) Provisional application No. 60/880,896, filed on Jan. 17, 2007.

(51) Int. Cl. A61N 1/05

(52) U.S. Cl. 607/118

(57) ABSTRACT

A method for treating a pulmonary condition in a subject includes one step of providing an implantable electrode assembly. The implantable electrode assembly includes a flexible member, at least one electrode operably secured to the flexible member, and an attachment mechanism for securing the at least one electrode to the flexible member. The implantable electrode assembly is implanted at a target site including an intraluminal location innervated by at least one nerve of the autonomic nervous system (ANS). The implantable electrode assembly is positioned at the target site such that the flexible member conforms to a portion of the inner surface of the target site, and the at least one electrode is positioned substantially adjacent the at least one nerve. Next, electric current is delivered to the at least one electrode to effect a change in the ANS of the subject.

### RELATED APPLICATIONS

This application claims priority from U.S. Provisional Application No. 60/880,896, filed Jan. 17, 2007, U.S. patent application Ser. No. 11/121,006, filed May 4, 2005, and U.S. patent application Ser. No. 11/222,766, filed Sep. 12, 2005. The subject matter of the aforementioned applications is incorporated herein by reference in their entireties.

### TECHNICAL FIELD

The present invention relates generally to apparatus and methods for treating pulmonary conditions, and more particularly to implantable medical devices and related methods for delivering electric current to a target site and effecting a change in the autonomic nervous system of a subject.

### BACKGROUND OF THE INVENTION

Diseases and disorders of the pulmonary system are among the leading causes of acute and chronic illness in the world. Pulmonary diseases or disorders may be organized into various categories, including, for example, breathing rhythm disorders, obstructive diseases, restrictive diseases, infectious diseases, pulmonary vasculature disorders, pleural cavity disorders, and others. Pulmonary dysfunction may involve symptoms such as apnea, dyspnea, changes in blood or respiratory gases, symptomatic respiratory sounds, e.g., coughing, wheezing, respiratory insufficiency, and/or general degradation of pulmonary function, among other symptoms.

A variety of methods are currently used to treat pulmonary diseases and disorders including, for example, the use of pharmacological compositions, such as albuterol, and surgical methods such as lung volume reduction surgery. Another method used to treat pulmonary disease and disorders involves electrostimulation of various nerves, such as the vagus and phrenic nerves, to modulate pulmonary function. Such electrostimulation methods, however, are often highly invasive and offer only short-term symptomatic relief.

### SUMMARY OF THE INVENTION

According to one aspect of the present invention, a method is provided for treating a pulmonary condition in a subject. One step of the method includes providing an implantable electrode assembly comprising a flexible member, at least one electrode operably secured to the flexible member, and an attachment mechanism for securing the at least one electrode to the flexible member. The implantable electrode assembly is implanted at a target site comprising an intraluminal location innervated by at least one nerve of the autonomic nervous system (ANS). The implantable electrode assembly is positioned at the target site such that the flexible member conforms to a portion of the inner surface of the target site, and the at least one electrode is positioned substantially adjacent the at least one nerve. Next, electric current is delivered to the at least one electrode to effect a change in the ANS of the subject.

According to another aspect of the present invention, an implantable electrode assembly is provided for positioning at a target site and for treating a pulmonary condition in a subject. The implantable electrode assembly comprises a flexible member having first and second end portions and oppositely disposed first and second surfaces. The first and second surfaces are for securing the flexible member at the target site. The implantable electrode assembly also comprises at least one electrode operably coupled to a portion of at least one of the first or second surfaces of the flexible member. The at least one electrode is adapted to selectively deliver electric current to the target site and effect a change in the ANS of the subject. Additionally, the implantable electrode assembly comprises an attachment mechanism for securing the at least one electrode to at least one of the first or second surfaces of the flexible member.

According to another aspect of the present invention, a method is provided for treating a pulmonary condition in a subject. One step of the method includes providing an implantable electrode assembly comprising a flexible member, at least one electrode operably secured to the flexible member, and an attachment mechanism for securing the at least one electrode to the flexible member. The implantable electrode assembly is implanted at a target site comprising an extraluminal location innervated by at least one nerve of the ANS. The implantable electrode is then positioned at the target site such that the flexible member envelops a portion of the target site and the at least one electrode is in direct contact with the at least one nerve. Next, electric current is delivered to the at least one electrode to effect a change in the ANS of the subject.

According to another aspect of the present invention, an implantable electrode assembly is provided for treating a pulmonary condition in a subject. The implantable electrode assembly comprises a clip member having first and second flexible arm members that are integrally formed with an attachment member. The flexible arm members are adapted for placement about a target site comprising an extraluminal location innervated by at least one nerve of the ANS. The implantable electrode assembly also comprises an electrode operably coupled to at least one of the first or second flexible arm members. The electrode is adapted to selectively deliver electric current to the target site and effect a change in the ANS of the subject.

According to another aspect of the present invention, a method is provided for treating a pulmonary condition in a subject. One step of the method includes providing an implantable electrode assembly. The implantable electrode assembly comprises a clip member having first and second flexible arm members that are integrally formed with an attachment member. The flexible arm members are adapted for placement about a target site comprising an extraluminal location innervated by at least one nerve of the ANS. The implantable electrode assembly also comprises an electrode operably coupled to at least one of the first or second flexible arm members. The electrode is adapted to selectively deliver electric current to the target site and effect a change in the ANS of the subject. The implantable electrode assembly is implanted at the target site and the positioned such that the first and second flexible arm members envelop a portion of the target site and the electrode is in direct contact with the at least one nerve. Next, electric current is delivered to the electrode to effect a change in the ANS of the subject.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1A is a perspective view of an implantable medical device constructed in accordance with the present invention;

FIG. 1B is a top plan view of the implantable medical device shown in FIG. 1A;

FIG. 2A is a schematic representation of the tracheo-bronchial tree;

FIG. 2B is a magnified schematic illustration showing a posterior view of the pulmonary plexus;

FIG. 2C is a magnified schematic illustration showing an anterior view of the pulmonary plexus;

FIG. 3 is a schematic illustration showing the sympathetic inputs of the pulmonary plexus from the sympathetic chain;

FIG. 4 is a schematic illustration showing the major nerves contributing to the pulmonary plexus;

FIG. 5A is a schematic illustration showing an implantable medical device similar to the one in FIG. 1A implanted at distal portion of the trachea adjacent the carina;

FIG. 5B is a schematic illustration showing an alternative embodiment of the implantable medical device in FIG. 1A implanted at the posterior pulmonary plexus;

FIG. 5C is a schematic illustration showing the implantable medical device in FIG. 1A implanted at the anterior pulmonary plexus;

FIG. 6A is a top plan view showing an alternative embodiment of the implantable medical device in FIG. 1A;

FIG. 6B is a perspective view of the implantable medical device shown in FIG. 6A;

FIG. 6C is a top plan view showing an alternative embodiment of the implantable medical device in FIG. 6A;

FIG. 7 is a schematic illustration showing the implantable medical device in FIG. 6A implanted at an anterior pulmonary plexus;

FIG. 8 is a perspective view showing an alternative embodiment of the implantable medical device in FIG. 1A;

FIG. 9 is a perspective view showing an alternative embodiment of the implantable medical device in FIG. 8;

FIG. 10 is a schematic illustration showing the implantable medical device in FIG. 8 implanted at an anterior pulmonary plexus; and

FIG. 11 showing the implantable medical device in FIG. 9 implanted at an anterior pulmonary plexus.

### DETAILED DESCRIPTION

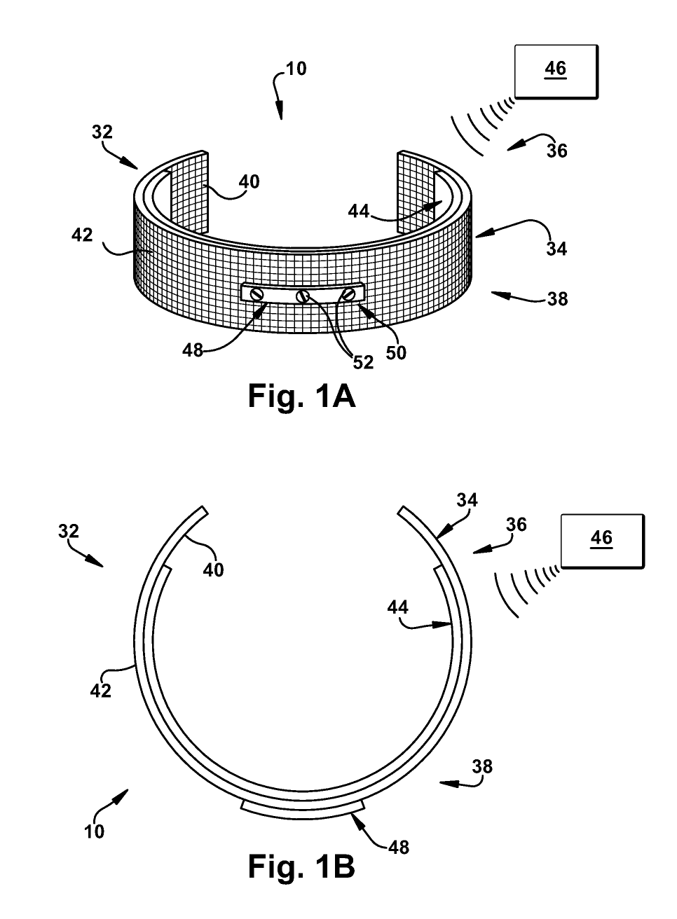

The present invention relates generally to apparatus and methods for treating pulmonary conditions, and more particularly to implantable medical devices and related methods for delivering electric current to a target site and effecting a change in the autonomic nervous system (ANS) of a subject. As representative of the present invention, FIGS. 1A and 1B illustrates an implantable medical device 10 for positioning at a target site and for treating a pulmonary condition in a subject.

Unless otherwise defined, all technical terms used herein have the same meaning as commonly understood by one of ordinary skill in the art to which the present invention pertains.

In the context of the present invention, the term “pulmonary condition” refers to both infection- and non-infection-induced disease and dysfunction of the respiratory system. Non-limiting examples of pulmonary conditions include genetic conditions, acquired conditions, and primary or secondary conditions which can include and/or may be caused by asthma, chronic obstructive pulmonary disease, cystic fibrosis, bronchiolitis, pneumonia, bronchitis, emphysema, adult respiratory distress syndrome, allergies, all types of lung cancer (e.g., small cell), including primary and metastatic cancers, brochiectasis, bronchopulmonary dysplasia, chronic bronchitis, chronic lower respiratory diseases, croup, high altitude pulmonary edema, pulmonary fibrosis, interstitial lung disease, reactive airway disease, lymphangioleiomyomatosis, neonatal respiratory distress syndrome, parainfluenza, pleural effusion, pleurisy, pneumothorax, primary pulmonary hypertension, psittacosis, pulmonary edema secondary to various causes, pulmonary embolism, pulmonary hypertension secondary to various causes, respiratory failure secondary to various causes, sleep apnea, sarcoidosis, smoking, stridor, acute respiratory distress syndrome, infectious diseases, such as SARS, tuberculosis, psittacosis infection, Q fever, parainfluenza and respiratory syncytial virus, and combinations thereof.

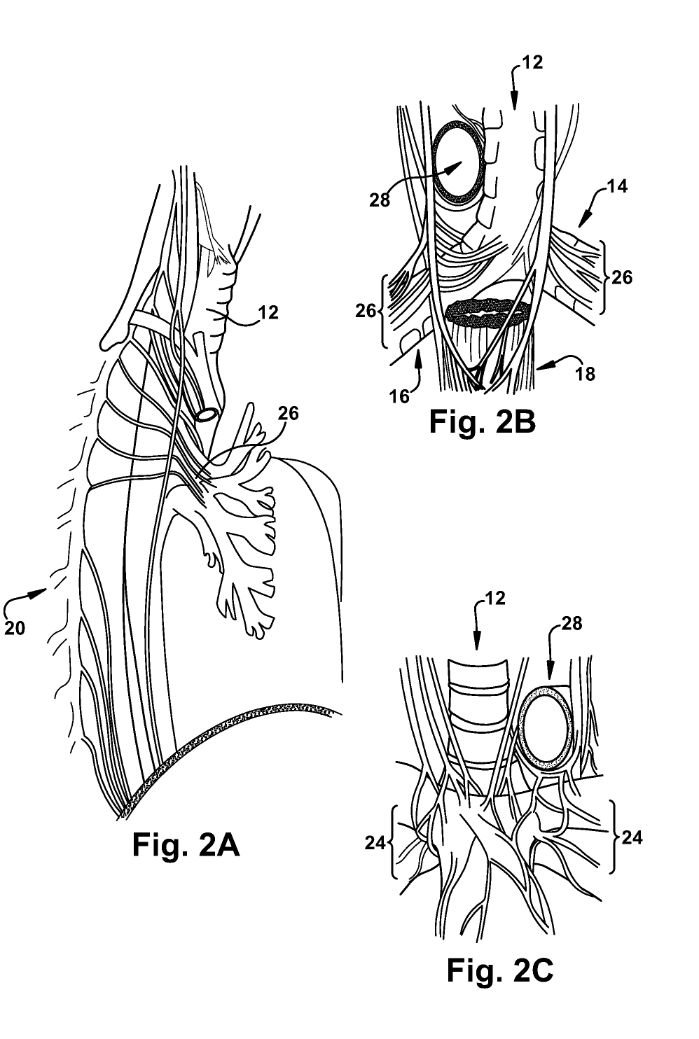

As used herein, the term “target site” refers to a desired anatomical location at which an implantable medical device 10 may be positioned. The target site can comprise a variety of anatomical locations, including intraluminal and extraluminal locations innervated by at least one nerve. For example, the target site can comprise an intravascular location innervated by at least one nerve. Alternatively, the target site can comprise an extraluminal location comprising at least one nerve. Target sites contemplated by the present invention are illustrated in FIGS. 2A-5C, FIG. 7, FIGS. 10-11, and are described in further detail below.

As used herein, the terms “modulate” or “modulating” refer to causing a change in neuronal activity, chemistry, and/or metabolism. The change can refer to an increase, decrease, or even a change in a pattern of neuronal activity. The terms may refer to either excitatory or inhibitory stimulation, or a combination thereof, and may be at least electrical, magnetic, thermal, ultrasonic, optical or chemical, or a combination of two or more of these. The terms “modulate” or “modulating” can also be used to refer to a masking, altering, overriding, or restoring of neuronal activity.

As used herein, the term “subject” refers to any warm-blooded organism including, but not limited to, human beings, pigs, rats, mice, dogs, goats, sheep, horses, monkeys, apes, rabbits, cattle, etc.

As used herein, the term “treating” refers to therapeutically regulating, preventing, improving, alleviating the symptoms of, and/or reducing the effects of a pulmonary condition.

A brief discussion of the neurophysiology is provided to assist the reader with understanding the present invention. The ANS regulates “involuntary” organs. The ANS includes the sympathetic nervous system (SNS) and the parasympathetic nervous system (PNS). The SNS is affiliated with stress and the “fight or flight response” to emergencies. The PNS is affiliated with relaxation and the “rest and digest response.” The ANS maintains normal internal function and works with the somatic nervous system. Autonomic balance reflects the relationship between parasympathetic and sympathetic activity. A change in autonomic balance is reflected in changes in heart rate, heart rhythm, contractility, remodeling, inflammation and blood pressure. Changes in autonomic balance can also be seen in other physiological changes, such as changes in abdominal pain, appetite, stamina, emotions, personality, muscle tone, sleep, and allergies, for example.

A more particular description of the neuroanatomy and neurophysiology of which the present invention pertains is presented below.

### Anatomy of Tracheo-Bronchial Tree

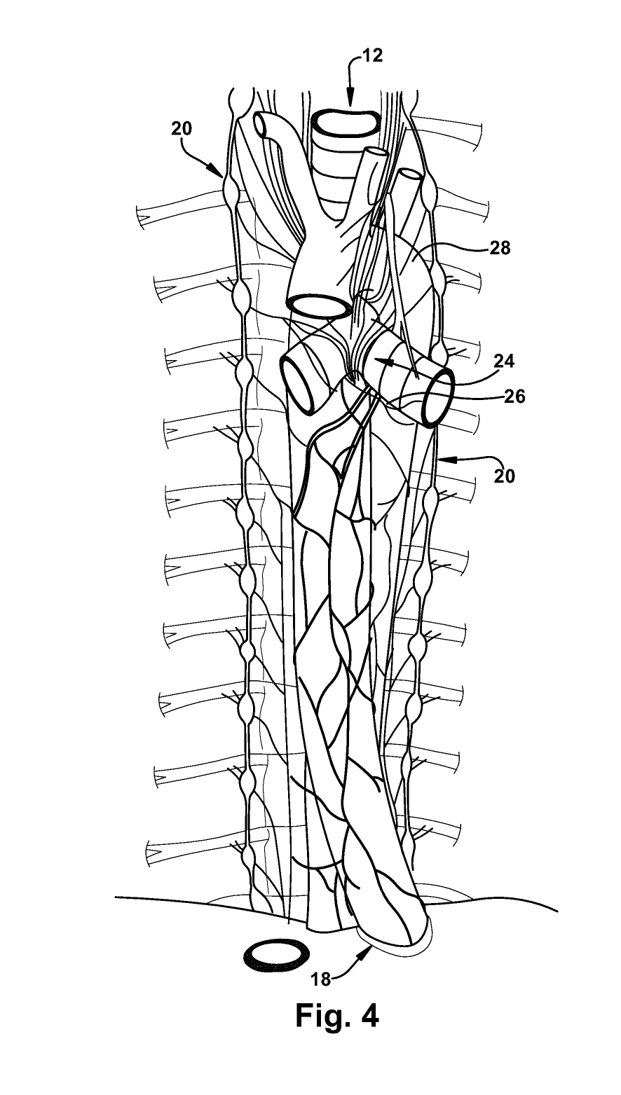

The trachea 12 (FIG. 2A) is a mobile cartilaginous and membranous tube that is part of the respiratory passage. It descends from the larynx, beginning at the level of C6, and then descends along the midline through the neck and thorax until it reaches its point of bifurcation at the level of T4. The trachea 12 is about 10 cm long and about 2 cm in diameter. It is covered by the pretracheal fascia. The walls of the trachea 12 are formed from fibrous tissue reinforced by the presence of 15-20 cartilaginous C-shaped rings. It is flattened posteriorly and supported along its 10- to 15-cm length by 16 to 20 horseshoe-shaped cartilaginous rings until bifurcating into right and left main bronchi 14 and 16 (FIG. 2B) at the level of the fifth thoracic vertebra. The cross-sectional area of the trachea 12 is considerably larger than that of the glottis, and may be more than 150 mm² and as large as 300 mm². This incompletion allows for the trachea 12 to lie on the esophagus 18 (FIG. 4) through its course.

In the neck, the trachea 12 lies anterior to the esophagus 18 with the recurrent laryngeal nerves situated laterally in the groove between the two. The trachea 12 lies posterior to the cervical fascia and the infrahyoid muscles, and anteriorly it is crossed by the isthmus of the thyroid gland and the jugular venous arch. Lateral to the trachea 12 are the lateral lobes of the thyroid gland, the inferior thyroid artery, and the carotid sheath. The trachea 12 receives its blood supply from the inferior thyroid arteries. Its lymph drains into the pretracheal and paratracheal lymph nodes. Nerve supply to the trachea 12 comes via the vagi, the recurrent laryngeal nerves, and the sympathetic trunks 20 (FIG. 2A).

### Nerve Supply of the Tracheo-Bronchial Tree and Respiratory System

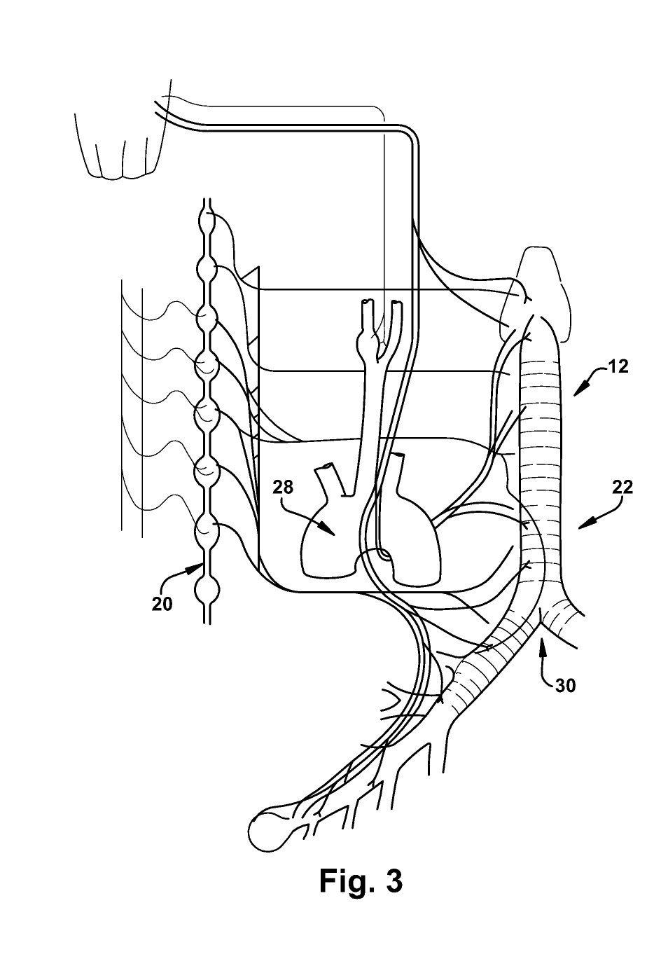

The tracheo-bronchial tree 22 (FIG. 3) and the lungs (not shown) are supplied from the anterior and posterior pulmonary plexuses 24 and 26 (FIGS. 2B and 2C), formed chiefly by branches from the sympathetic and vagus nerves and collectively referred to as the pulmonary plexus. The filaments from the anterior and posterior plexuses 24 and 26 accompany the bronchial tubes, supplying efferent fibers to the bronchial muscle and afferent fibers to the bronchial mucous membrane and probably to the alveoli of the lung. Small ganglia are found upon these nerves. The pulmonary plexus thus has three major inputs coming from sympathetic ganglia, parasympathetic ganglia, and the cardiac plexus.

### Sympathetic Component of the Pulmonary Plexus

The thoracic portion of the sympathetic trunk 20 consists of a series of ganglia, which usually correspond in number to that of the vertebrae; but, on account of the occasional coalescence of two ganglia, their number is uncertain. The thoracic ganglia rest against the heads of the ribs and are covered by the costal pleura; the last two, however, are more anterior than the rest and are placed on the sides of the bodies of the eleventh and twelfth thoracic vertebrae. The ganglia are small in size and of a grayish color. The first, which is larger than the others, is of an elongated form and frequently blended with the inferior cervical ganglion. The ganglia are connected together by the intervening portions of the sympathetic trunk 20. Two rami communicantes, a white and a gray, connect each ganglion with its corresponding spinal nerve. The branches from the upper five thoracic ganglia are very small and supply filaments to the thoracic aorta and its branches. Branches from the second, third, fourth and fifth (occasionally sixth and seventh) ganglia enter the posterior pulmonary plexus 26. The branches from the lower seven thoracic ganglia are large, white in color, and distribute filaments to the aorta, thereby uniting to form the greater, lesser, and the lowest splanchnic nerves.

### Parasympathetic Components of the Pulmonary Plexus

Parasympathetic innervations come through the vagal branches that contribute to the pulmonary plexus and include the anterior and posterior bronchial branches. The anterior bronchial branches (rami bronchiales anteriores and anterior or ventral pulmonary branches) are two or three in number, of small size, and are distributed on the anterior surface of the root of the lung. They join with sympathetic to form the anterior pulmonary plexus 24.

The posterior bronchial branches (rami bronchiales posteriors and posterior or dorsal pulmonary branches) are more numerous and larger than the anterior bronchial branches, and are distributed on the posterior surface of the root of the lung. They are joined by filaments from the third and fourth (sometimes also from the first and second) thoracic ganglia of the sympathetic trunk 20, and form the posterior pulmonary plexus 26. Branches from this plexus 26 accompany the ramifications of bronchi through the substance of the lung.

### Inputs From the Cardiac Plexus

The cardiac plexus is situated at the base of the heart and is divided into a superficial part, which lies in the concavity of the aortic arch 28 (FIG. 3), and a deep part located between the aortic arch and the trachea 12. The two parts are closely connected. The superficial part of the cardiac plexus lies beneath the aortic arch 28 and in front of the right pulmonary artery. The cardiac plexus is formed by the superior cardiac branch of the left sympathetic and the lower superior cervical cardiac branch of the left vagus. A small ganglion, the cardiac ganglion of Wrisberg, is occasionally found connected with these nerves at their junction point. The superficial part of the cardiac plexus gives branches to the deep part of the plexus, to the anterior coronary plexus, and to the left anterior pulmonary plexus 24.

The deep part of the cardiac plexus is situated in front of the bifurcation of the trachea 12, known as the carina 30 (FIG. 3), above the point of division of the pulmonary artery, and behind the aortic arch 28. It is formed by the cardiac nerves derived from the cervical ganglia of the sympathetic and the cardiac branches of the vagus and recurrent nerves. The only cardiac nerves which do not enter into the formation of the deep part of the cardiac plexus are the superior cardiac nerve of the left sympathetic and the lower of the two superior cervical cardiac branches from the left vagus, which pass to the superficial part of the plexus.

Branches from the right half of the deep part of the cardiac plexus pass in front of, and others behind, the right pulmonary artery. Those that pass in front are more numerous, transmit a few filaments to the anterior pulmonary plexus 24, and continue onward to form part of the anterior coronary plexus. Those behind the pulmonary artery distribute a few filaments to the right atrium and then continue onward to form part of the posterior coronary plexus. The left half of the deep part of the plexus is connected with the superficial part of the cardiac plexus, gives filaments to the left atrium and to the anterior pulmonary plexus 24, and then continues to form the greater part of the posterior coronary plexus.

### Structural and Functional Divisions of the Pulmonary Plexus

The pulmonary plexus is divided into the anterior and posterior divisions 24 and 26. The anterior part 24 lays over the carina 30 near the superior aspect of the pulmonary trunk and behind the aortic arch 28. The posterior part 26 lies on the posterior wall of trachea 12 between the trachea and esophagus 18. The anterior part 24 is primarily sympathetic while the posterior part 26 is primarily parasympathetic. While the anterior and posterior pulmonary plexuses 24 and 26 are primarily sympathetic and parasympathetic (respectively), the plexuses are not entirely separate and, rather, are mixed entities.

Once the pulmonary plexus enters the tracheo-bronchial tree 22, it further divides into peribronchial and perivascular parts. The peribronchial plexus is mainly formed by the branches from the recurrent laryngeal and vagus nerves, dividing and rejoining to form a wide-meshed plexus on the outer sides of the cartilages. This network, containing a few small ganglia, is inconspicuous anteriorly, but is well marked posteriorly where it lies on the external elastic lamina.

Filaments from the posterior pulmonary plexus 26 pass into the external elastic lamina of the trachea 12 and form, just behind the trachealis muscle, a well-defined longitudinal chain of nerves with scattered ganglia. A wider-meshed plexus, with small ganglia at some of its nodes, is formed in the substance of the trachealis muscle. This could be termed a “primary plexus,” for within its meshes is found a finer “secondary plexus” which, in turn, contributes to the still finer fibers of a “tertiary plexus” running parallel to the muscle fibers.

The primary plexus extends in depth through the substance of the trachealis muscle and eventually appears in the tissues of the submucosa. Besides sinking through the muscle, this plexus provides further lateral branches which merge imperceptibly with the fibers which run between and internal to the cartilage plates. Thus, there is a plexus in the muscle continuous with a plexus inside and between the cartilage plates. This plexus is continuous with the nerves of the submucosa anterior to the trachealis muscle.

### Function of the Sympathetic and Parasympathetic Divisions

Sympathetic, parasympathetic, non-adrenergic, and non-cholinergic pathways innervate airway smooth muscle and can produce either bronchoconstriction or bronchodilation when they are activated or inhibited. Therefore, the ANS plays a primary role in regulating airway caliber, and its dysfunction is likely to contribute to the pathogenesis of airways diseases. Indeed, parasympathetic activity is known to promote bronchoconstriction of the airways, and an alteration of muscarinic receptors could lead to an increase of airway hyperresponsiveness (AHR) and then to bronchoconstriction. Moreover, airway inflammation, which is a characteristic feature of bronchial asthma, might alter both the contractile properties and the autonomic regulation of airway smooth muscle. These findings support the hypothesis that autonomic dysfunction and/or dysregulation contributes to the pathogenesis of AHR.

Airway tone is influenced by cholinergic neural mechanisms, adrenergic mechanisms, and by more recently described neural mechanisms which are non-adrenergic and non-cholinergic (NANC). Sympathetic innervation in human airways is to the smooth muscle and through ganglia to submucosal glands and bronchial vessels. Airway tone may also be influenced by circulating adrenaline, and there is some evidence that adrenaline secretion may be impaired in asthma. Beta-adrenoceptors (which are almost entirely of the beta 2-subtype) are localized to many cell types in airways, and beta-agonist may be beneficial in airway obstruction by not only directly relaxing airway smooth muscle (from trachea to terminal bronchioles), but also by inhibiting mast cell mediator release, modulating cholinergic nerves, reducing bronchial edema, and by reversing defects in mucociliary clearance.

Alpha-adrenoceptors, which are bronchoconstrictor, may be activated by inflammatory mediators and disease, and alpha-agonists cause bronchoconstriction in asthmatic patients. However, alpha-antagonists have little effect, which questions the role of alpha-receptors in asthma. NANC nerves which relax human airways have been demonstrated in vitro. Although the neurotransmitter is not certain, there is now convincing evidence that it may be vasoactive intestinal peptide (VIP) and a related peptide histidine methionine (PHM). VIP and PHM immuno-active nerves are found in human airways, and both peptides potently relay human airways in vitro.

One embodiment of the present invention is illustrated in FIGS. 1A and 1B. In FIGS. 1A and 1B, the implantable medical device 10 can comprise an implantable electrode assembly 32 including a flexible member 34 having first and second end portions 36 and 38 and oppositely disposed first and second surfaces 40 and 42. As described in further detail below, the particular geometry and flexible properties of the flexible member 34 allow the implantable electrode assembly 32 to be securely positioned at an extraluminal or intraluminal target site.

The flexible member 34 may be comprised of a flexible, biocompatible material, such as a polypropylene mesh. Other examples of suitable materials include DACRON (Invista, Wichita, Kans.), GORETEX (W. L. Gore & Associates, Flagstaff, Ariz.), woven velour, polyurethane, or heparin-coated fabric, graphite, ceramic, and hardened plastics. The flexible member 34 may also be made of a biocompatible, medical grade metal or metal alloy, such as cobalt-nickel alloys (e.g., Elgiloy), titanium, nickel-titanium alloys (e.g., Nitinol), cobalt-chromium alloys (e.g., Stellite), nickel-cobalt-chromium-molybdenum alloys (e.g., MP35N), and stainless steel.

The flexible member 34 may have a C-shaped geometry as shown in FIGS. 1A and 1B, or any other suitable geometry, such as a U- or V-shaped geometry. Alternatively, the flexible member 34 may have a complete ring or O-shaped configuration. It will be appreciated that the flexible member 34 may have any dimension (e.g., width, length, circumference, etc.) as required by a particular application of the implantable electrode assembly 32.

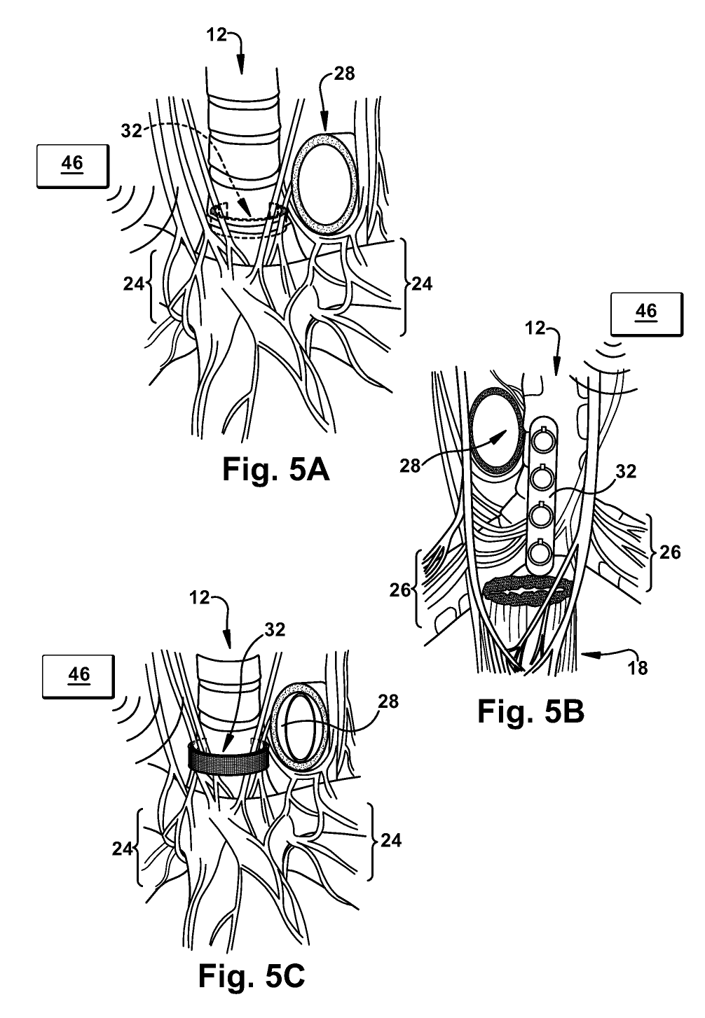

The implantable electrode assembly 32 also includes at least one electrode 44 for delivering an electric current to a target site. The electrode 44 is operably coupled to a portion of the first surface 40 or the second surface 42 of the flexible member 34, and has a thin, flattened configuration. For example, the electrode 44 is operably coupled to the first surface 40 of the flexible member 34 as shown in FIGS. 1A and 1B. It will be appreciated that the electrode 44 may have any shape and size including, for example, a triangular shape, a rectangular shape, an ovoid shape, and/or a band-like shape (e.g., a split band configuration), and is not limited to the shape and size illustrated in FIGS. 1A and 1B. For example, the electrode 44 can have any size from about 5 degrees to about 360 degrees, and may be wedge-shaped, pointed, rounded, etc. It will also be appreciated that the electrode 44 can comprise a ½ or ¼ ring configuration, a plate electrode, a paddle electrode (FIG. 5B), a cuff electrode, a cylindrical electrode, or the like.

The electrode 44 (FIGS. 1A and 1B) may be configured so that the implantable electrode assembly 32 has a unipolar construction using surround tissue as a ground or, alternatively, a multipolar construction using leads (not shown) connected to a portion of the implantable electrode assembly. The electrode 44 may be made of any material capable of conducting an electrical current, such as titanium, platinum, platinum-iridium, or the like.

As shown in FIGS. 1A and 1B, the electrode 44 extends across only a portion of the first surface 40 of the flexible member 34. It will be appreciated, however, that any portion of the first surface 40, such as the entire first surface, may be covered by the electrode 44. To facilitate focal delivery of electric current to a target site, the size and shape of the electrode 44 may be varied as needed. Additionally or optionally, the entire surface area of the electrode 44 may be conductive or, alternatively, only a portion of the surface area of the electrode may be conductive. By modifying the size, shape, and conductivity of the surface of the electrode 44, the surface area of the electrode that contacts a target site may be selectively modified to facilitate focal delivery of electric current. For example, electric current can be delivered to the electrode 44 such that the electric current is conducted only through selective portions of the electrode. Delivery of electric current can then be selectively controlled or “titrated” to achieve a desired physiological effect.

Electric current can be delivered to the implantable electrode assembly 32 using a variety of internal, passive, or active energy delivery sources 46. The energy delivery source 46 may include, for example, radio frequency (RF) energy, X-ray energy, microwave energy, acoustic or ultrasound energy, such as focused ultrasound or high intensity focused ultrasound energy, light energy, electric field energy, thermal energy, magnetic field energy, combinations of the same, or any other energy delivery source used with implantable pulse generators known in the art. As shown in FIG. 1A, for example, an RF energy delivery source 46 may be wirelessly coupled to the implantable electrode assembly 32. Alternatively, the energy delivery source 46 may be directly coupled to the implantable electrode assembly 32 using an electrical lead.

Electric current can be delivered to the implantable electrode assembly 32 continuously, periodically, episodically, or a combination thereof. For example, electric current can be delivered in a unipolar, bipolar, and/or multipolar sequence or, alternatively, via a sequential wave, charge-balanced biphasic square wave, sine wave, or any combination thereof. Where a plurality of electrodes 44 are included as part of the implantable electrode assembly 32, electric current can be delivered to all the electrodes at once or, alternatively, to only a select number of desired electrodes using a controller (not shown) and/or complex practices, such as current steering.

The particular voltage, current, and frequency delivered to the implantable electrode assembly 32 may be varied as needed. For example, electric current can be delivered to the implantable electrode assembly 32 at a constant voltage (e.g., at about 0.1 v to about 25 v), at a constant current (e.g., at about 25 microampes to about 50 milliamps), at a constant frequency (e.g., at about 5 Hz to about 10,000 Hz), and at a constant pulse-width (e.g., at about 50 μsec to about 10,000 μsec).

As noted above, delivery of electric current to the electrode 44 may be accomplished via a controller (not shown) operably coupled to the implantable electrode assembly 32. The controller may comprise an electrical device which operates like a router by selectively controlling delivery of electric current to the electrode 44. For example, the controller may vary the frequency or frequencies of the electric current being delivered to the electrode 44. By selectively controlling delivery of electric current to the electrode 44, the controller can facilitate focal delivery of electric current to a target site.

Typically, delivery of electric current to the implantable electrode assembly 32 results in activation of at least one nerve at a target site which, in turn, effects a change in the ANS of a subject. Alternatively, deactivation or modulation of electric current to the implantable electrode assembly 32 may cause or modify the activity of at least one nerve at a target site. For example, electric current may be delivered to the implantable electrode assembly 32 and consequently inhibit activation of at least one nerve at, adjacent to, and/or distant from a target site. Modulating the electric current delivered to the implantable electrode assembly 32 may induce a change or changes in the activity, chemistry, and/or metabolism of at least one nerve directly or indirectly associated with a target site.

It should be appreciated, however, that means other than, or in addition to, electric current, such as chemical or biological means, may also be delivered to a target site and thereby effect a change in the ANS. For example, any of the implantable medical devices 10 described herein may include at least one therapeutic agent for eluting into the parenchymal tissue, vascular tissue and/or blood stream. The therapeutic agent may be capable of preventing a variety of pathological conditions including, but not limited to, thrombosis, stenosis and inflammation. Accordingly, the therapeutic agent may include at least one of an anticoagulant, an antioxidant, a fibrinolytic, a steroid, an anti-apoptotic agent, an anti-inflammatory agent, a receptor agonist or antagonist, a hormone, a neurotransmitter, and/or modulatory neurotoxins, such as botox.

Optionally or additionally, the therapeutic agent may be capable of treating or preventing other diseases or disease processes, such as microbial infections, for example. In these instances, the therapeutic agent may include an anti-microbial agent and/or a biological agent such as a cell, peptide, or nucleic acid. The therapeutic agent can be simply linked to a surface of an implantable medical device 10, embedded and released from within polymer materials, such as a polymer matrix, or surrounded by and released through a carrier.

Referring again to FIGS. 1A and 1B, the electrode 44 is operably coupled to the first surface 40 of the flexible member 34 by an attachment mechanism 48. The attachment mechanism 48 can comprise a metal member 50 operably coupled to the second surface 42 of the flexible member 34. Alternatively, where the electrode 44 is operably coupled to the second surface 42 of the flexible member 34, the metal member 50 can be operably coupled to the first surface 40 of the flexible member. As shown in FIG. 1A, the metal member 50 has a plurality of securing members 52 extending radially therethrough. The securing members 52 extend through the flexible member 34 and secure the electrode 44 to the first surface 40 of the flexible member. It will be appreciated that the attachment mechanism 48 may include a variety of other devices and mechanisms for securing the electrode 44 to the flexible member 34. For example, screws, clips, pins, adhesives (e.g., fibrin glue), staples, and/or biological membranes may be used. Alternatively, a magnetic mechanism (not shown) may also be used to secure the electrode 44 to the flexible member 34.

Any of the implantable medical devices 10 described herein may also include a layer of biocompatible material (not shown) to facilitate biocompatibility of the implantable medical device. The layer of biocompatible material may be synthetic such as DACRON, GORETEX, woven velour, polyurethane, or heparin-coated fabric. Alternatively, the layer of biocompatible material may be a biological material such as bovine or equine pericardium, peritoneal tissue, an allograft, a homograft, patient graft, or a cell-seeded tissue. The biocompatible layer can cover the entire implantable medical device 10 or, alternatively, may be attached in pieces or interrupted sections to facilitate placement of the implantable medical device.

The implantable medical devices 10 described herein can be part of an open- or closed-loop system. In an open-loop system, for example, a physician or subject may, at any time, manually or by the use of pumps, motorized elements, etc. adjust treatment parameters such as pulse amplitude, pulse width, pulse frequency, or duty cycle. Alternatively, in a closed-loop system, electrical parameters may be automatically adjusted in response to a sensed symptom or a related symptom indicative of the extent of the pulmonary condition being treated. In a closed-loop feedback system, a sensor (not shown) that senses a condition (e.g., a metabolic parameter of interest) of the body can be utilized. More detailed descriptions of sensors that may be employed in a closed-loop system, as well as other examples of sensors and feedback control techniques that may be employed are disclosed in U.S. Pat. No. 5,716,377, which is hereby incorporated by reference in its entirety.

Although described in more detail below, it should be appreciated that incorporating an implantable medical device 10 as part of a closed-loop system can include placing an implantable medical device in a blood vessel adjacent a target site, detecting a bodily activity associated with a pulmonary condition, and then activating the implantable medical device to apply electric current to the target site in response to the detected bodily activity. Such bodily activity can include any characteristic or function of the body, such as respiratory function (e.g., respiratory rate), body temperature, blood pressure, metabolic activity such as fluid glucose levels, hormone levels, and/or nitrogen, oxygen and/or carbon dioxide levels, cerebral blood flow, pH levels (e.g., in blood, tissue, and other bodily fluids), galvanic skin responses (e.g., perspiration), electrocardiogram, muscle tone in the diaphragm and other muscles, electroencephalogram, nerve action potential, body movement, response to external stimulation, speech, motor activity, ocular activity, cognitive function, and the like.

The analysis of constituents of breath, for example, provides an easily accessible, non-invasive method of monitoring inflammation as a number of by-products of airway inflammation and oxidative stress are found in exhaled air. Accordingly, a closed-loop system can include a sensor for detecting at least one metabolic parameter associated with pulmonary inflammation from the exhaled vapor of a subject. Examples of the metabolic parameter can include, but are not limited to, eicosanoids (e.g., 8-isoprostanes, leukotriene₄ (LTE₄), LTC₄, LTD₄, LTB₄, PG, TX), NO-related products (e.g., nitrotyrosine, NO₂⁻/NO₃⁻, S-nitrosothiols), hydrogen peroxide, lipid peroxidation products, vasoactive amines, ammonia, cytokines (e.g., IL-1β, IL-2, IL-6, TNF-α, IL-8), and electrolytes (e.g., Na, Cl, Mg, Ca).

In another embodiment of the present invention, methods are provided for treating a pulmonary condition in a subject. The methods of the present invention can include an indirect approach, a direct approach, or combinations thereof for treating a pulmonary condition. By “direct” it is meant that an implantable medical device 10 is placed on or near at least one nerve capable of effecting a change in the ANS. By “indirect” it is meant that an implantable medical device 10 is placed at an intraluminal target site (e.g., a blood vessel, soft tissue, the trachea 12, bronchi, the esophagus 18, etc.) which is innervated by at least one nerve capable of effecting a change in the ANS.

As described in more detail below, the pulmonary condition may be treated by electrically modulating the SNS, the PNS, or both. By “electrically modulating,” it is meant that at least a portion of the ANS is altered or changed by electrical means. Electrical modulation of the ANS may affect central motor output, nerve conduction, neurotransmitter release, synaptic transmission, and/or receptor activation. For example, at least a portion of the ANS may be electrically modulated to alter, shift, or change parasympathetic function from a first state to a second state, where the second state is characterized by an increase or decrease in parasympathetic function relative to the first state. Alternatively, at least a portion of the ANS may be electrically modulated to alter, shift, or change sympathetic function from a first state to a second state, where the second state is characterized by an increase or decrease in sympathetic function relative to the first state.

It will be appreciated that delivering electrical energy to a target site can modulate the ANS in any desirable combination of ways, including, for example, increasing both parasympathetic and sympathetic function, increasing parasympathetic function while decreasing sympathetic function, decreasing both parasympathetic and sympathetic function, decreasing parasympathetic function while increasing sympathetic function, increasing parasympathetic function without any effect on sympathetic function, decreasing parasympathetic function without any effect on sympathetic function, increasing sympathetic function without any effect on parasympathetic function, and/or decreasing sympathetic function without any effect on parasympathetic function.

Depending upon a particular application (i.e., a particular pulmonary condition), the target site can include a variety of anatomical locations, such as intraluminal and extraluminal locations innervated by at least one nerve of the ANS. Non-limiting examples of target sites include the esophagus 18, the tracheo-bronchial tree 22, blood vessels, intraluminal or extraluminal sites proximal to the pulmonary plexus, at the pulmonary plexus, and/or distal to the pulmonary plexus.

Non-limiting examples of nerves which can innervate the target site and which can effect a change in the ANS include a spinal nerve, a preganglionic fiber of a spinal nerve, a postganglionic fiber of a spinal nerve, a sympathetic chain ganglion, a thoracic sympathetic chain ganglion, a superior cervical ganglion, a cervical ganglion, a lower cervical ganglion, an inferior cervical ganglion, an intramural ganglion, a splanchnic nerve, an esophageal plexus, a cardiac plexus, a pulmonary plexus, an anterior pulmonary plexus 24, a posterior pulmonary plexus 26, a celiac plexus, a hypogastric plexus, an inferior mesenteric ganglion, a celiac ganglion, and a superior mesenteric ganglion.

Target sites proximal to the pulmonary plexus include the thoracic sympathetic trunk 20. Target sites at the pulmonary plexus include the anterior pulmonary plexus 24, in which case delivery of electrical energy to the target site will primarily modulate the SNS. Another target site at the pulmonary plexus includes the posterior pulmonary plexus 26, in which case delivery of electrical energy to the target site will primarily modulate the PNS. Target sites distal to the pulmonary plexus include the intramuscular and subchondrial plexuses, in which case delivery of electrical energy to the target site will primarily modulate the PNS.

In an example of the present invention, an indirect approach to treating a subject with a pulmonary condition (e.g., asthma) is provided. One step of the method includes providing an implantable medical device 10 that is similarly constructed as the implantable electrode assembly 32 illustrated in FIGS. 1A and 1B. As shown in FIG. 5A, for example, the electrode 44 of the implantable electrode assembly 32 can be operably coupled to the second surface 42 of the flexible member 34 via the attachment mechanism 48. The implantable electrode assembly 32 can be implanted at a desired intraluminal target site innervated by at least one nerve of the ANS. For example, the target site can comprise a distal portion of the trachea 12 adjacent the carina 30 which is innervated by a portion of the anterior pulmonary plexus 24.

Prior to implanting the implantable electrode assembly 32, the anatomical dimensions of the target site can be determined using known methods, such as MRI, CT, or visual inspection via endoscopy. After determining the dimensions of the target site, an appropriately-sized implantable electrode assembly 32 is selected for implantation. Any one or combination of known surgical approaches can be used to implant the implantable electrode assembly 32. Examples of suitable approaches include, but are not limited to, trans-tracheal, trans-mediastinal, transvenous (e.g., through the pulmonary trunk), trans-aortic, trans-esophageal, trans-thoracic, percutaneous, posterior para-spinal, anterior transcutaneous, subcutaneous, and transmediastinal routes. To facilitate placement of the implantable electrode assembly 32 at the target site, a portion of the implantable electrode assembly may be made of a radio-opaque material or include radio-opaque markers (not shown) to facilitate fluoroscopic visualization.

Depending upon the orientation of the implantable electrode assembly 32 at the target site, such as the pulmonary plexus, the SNS, PNS, or a combination thereof can be selectively modulated. For example, if delivery of electric current is made posteriority, then the PNS can be selectively modulated depending upon the anatomical structure and physiological innervation at the target site. Alternatively, if delivery of electric current is made anteriorily, then the SNS can be selectively modulated depending upon the anatomical structure and physiological innervation at the target site. The implantable electrode assembly 32 can thus modulate different parts of pulmonary plexus depending on the location of the implantable electrode assembly. It should be appreciated that electric current can be delivered both anteriorily and posteriority through the use of more than one implantable electrode assembly 32.

Where an endoscopic approach (e.g., bronchoscopy) is used to deliver the implantable electrode assembly 32, the implantable electrode assembly is positioned at the target site such that a portion of the electrode 44 is placed in contact with an anterior portion of distal trachea 12 (FIG. 5A). It will be appreciated, however, that the implantable electrode assembly 32 can be positioned at the target site such that a portion of the electrode 44 contacts the internal tracheal wall at any portion (e.g., anterior, posterior, right, left) and at any degree or angle (e.g., from about 5 degrees to about 360 degrees). After the implantable electrode assembly 32 has been appropriately positioned at the target site, electric current is delivered to the implantable electrode assembly. As shown in FIG. 5A, RF energy is delivered to the implantable electrode assembly 32 via a wirelessly-coupled energy delivery source 46. The electric current can then be directed to the electrode 44 via a controller which directs and apportions a desired amount of electric current to the electrode. As electric current is delivered to the electrode 44, the electrode conducts electric current through the anterior tracheal wall to the anterior pulmonary plexus 24. For example, electrical stimulation or activation of the anterior pulmonary plexus 24 increases sympathetic nerve function, in turn causing the bronchioles to dilate and thus treat the asthmatic symptoms of the subject.

It should be appreciated that the implantable medical device 10 can additionally or alternatively be implanted at an intravascular target site to effect a change in the ANS of a subject. For example, various nerves extend around the aortic arch 28, the vagus nerve extends past the ligamentum arteriosum, the anterior pulmonary plexus 24 crosses the left pulmonary artery, and the right vagus nerve extends past a subclavian artery and the cupola of pleura. Cardiac nerves also extend past the brachiocephalic trunk near the trachea 12 and the arch of an azygous vein to the right pulmonary artery. Accordingly, an implantable medical device 10 can be implanted percutaneously, for example, in the left pulmonary artery and then electric current delivered to the implantable medical device to modulate the pulmonary plexus through an arterial or venous wall. It should additionally be appreciated that the implantable medical device 10 may be placed at an intraluminal or extraluminal location on or about an organ of the gastrointestinal and/or genitourinary system.

In another example of the present invention, a direct approach to treating a subject pulmonary condition (e.g., asthma) is provided. One step of the method can include providing the implantable medical device 10 shown in FIG. 5B. In FIG. 5B, the implantable medical device 10 can comprise a series of implantable electrode assemblies 32 arranged in series or a paddle-like configuration. The implantable electrode assembly 32 can be implanted at a target site in the sympathetic trunk 20, for example, using an approach similar to the one taken for endoscopic thoracic sympathectomy or a posterior para-spinal approach. For example, under general anesthesia and using single lumen tracheal intubation, a subject can be placed in semi-Fowler's position with his or her arms abducted. Two ports can then be made. One port can be in the middle or posterior axillary line at the level of the nipple. The lung can then be gently pushed down and a small endoscope (not shown) (e.g., a long narrow tube with a light source and lens) inserted into the left chest. The scope can be inserted between the ribs and the chest space while being monitored on a video monitor.

The second port can then be made in the axilla for insertion of a hooked diathermy probe or an endoscopic clip-applicator. The sympathetic trunk 20 can be visulaized through the thoracoscope. Using the video as a guide, the implantable electrode assembly 32 can be positioned at the target site. The implantable electrode assembly 32 can be placed over a portion of the sympathetic trunk 20 so that at least one nerve of the sympathetic trunk is covered by a portion of each electrode 44 of the implantable electrode assembly. The implantable electrode assembly 32 can be placed using a variety of known methods including, for example, by use of endoscopic clips (not shown). Depending upon the target site, the closing pressure of the clips can be adjusted as needed.

After the implantable electrode assembly 32 is securely positioned about the sympathetic trunk 20, electric current can be delivered to the implantable electrode assembly. The electric current can then be directed to the electrode 44 via a controller which directs and apportions a desired amount of electric current to the electrode. Delivery of electric current can modulate the activity of the sympathetic trunk 20. For example, the sympathetic trunk 20 may be stimulated, in turn causing the bronchioles of the lungs to dilate and reduce and/or eliminate the asthmatic symptoms in the subject.

In another example of the present invention, a direct approach may be used to treat a subject with a pulmonary condition, such as asthma. One step of the method can include providing the implantable electrode assembly 32 shown in FIGS. 1A and 1B. The implantable electrode assembly 32 can be implanted at a target site comprising the anterior pulmonary plexus 24. Various surgical and/or percutaneous approaches may be used to access the target site. Examples of suitable approaches include, but are not limited to, trans-tracheal, trans-mediastinal, transvenous (e.g., through the pulmonary trunk), trans-aortic, trans-esophageal routes, trans-thoracic, percutaneous, posterior para-spinal, anterior transcutaneous, subcutaneous, and transmediastinal routes.

Where a trans-mediastinal approach is used, for example, an anterior mediastinoscopy or thoracoscopy may be used to place the implantable electrode assembly 32. The advantage of thoracoscopy is the visibility, even to the subcarinal anterior mediastinum. The mediastinoscopy can be performed under general anesthesia using a standard KARL STORZ mediastinoscope (Culver City, Calif.), either with or without video assistance. An incision can be made in the suprasternal notch and dissection performed caudally to the thyroid isthmus and then continued by blunt dissection into the pretracheal space. Once the carina 30 is identified, the implantable electrode assembly 32 can be placed around the distal-most part of trachea 12 above the surrounding soft tissue so that a portion of the flexible member 34 envelops a portion of the distal trachea which is innervated by the anterior pulmonary plexus 24. More particularly, the implantable electrode assembly 32 can be positioned at the target site such that a portion of the electrode 44 is placed in direct contact with a portion of the anterior pulmonary plexus 24 (FIG. 5C).

It will be appreciated, however, that the implantable electrode assembly 32 can be positioned at the target site such that a portion of the electrode 44 contacts the external tracheal wall at any portion (e.g., anterior, posterior, right, left) and at any degree or angle (e.g., from about 5 degrees to about 360 degrees). After the implantable electrode assembly 32 has been appropriately positioned at the target site, electric current can be delivered to the implantable electrode assembly. It should be appreciated that an alternative implantation approach could include transillumination of the skin via a bronchoscopy device (not shown). Using such an approach, the bronchoscopy device could delineate the target site via a light. The light, in turn, could trans-illuminate the trachea 12 and bronchi in a dorsal direction. Additionally or optionally, the light could further serve as a percutaneous guide when approaching the target site.

As shown in FIG. 5C, RF energy can be delivered to the implantable electrode assembly 32 via a wirelessly-coupled energy delivery source 46. The electric current can then be directed to the electrode 44 via a controller which directs and apportions a desired amount of electric current to the electrode. As electric current is delivered to the electrode 44, the electrode can conduct electric current through the anterior tracheal wall to the anterior pulmonary plexus 24. For example, electrical stimulation or activation of the anterior pulmonary plexus 24 can increase sympathetic nerve function, in turn causing the bronchioles to dilate and thus treat the asthmatic symptoms of the subject.

An electrical stimulus regimen comprising a desired temporal and spatial distribution of electric current to a target site may be selected to promote long term efficacy of the present invention. It is contemplated that uninterrupted or otherwise unchanging activation of the target site may result in the at least one nerve present at the target site to become less responsive over time, thereby diminishing the long term effectiveness of the therapy. Therefore, the electrical stimulus regimen maybe selected to activate, deactivate, or otherwise modulate the implantable medical devices 10 described herein in such a way that therapeutic efficacy is maintained for a desired period of time.

In addition to maintaining therapeutic efficacy over time, the electrical stimulus regimen may be selected to reduce the power requirement/consumption of the implantable medical devices 10 described herein. For example, the electrical stimulus regimen may dictate that an implantable medical device 10 be initially activated at a relatively higher energy and/or power level, and then subsequently activated at a relatively lower energy and/or power level. The first level attains the desired initial therapeutic effect, and the second (lower) level sustains the desired therapeutic effect long term. By reducing the energy and/or power levels after the desired therapeutic effect is initially attained, the energy required or consumed by an implantable medical device 10 is also reduced long term.

It should be appreciated that unwanted collateral stimulation of tissues and/or nerves adjacent the target site may be limited by creating localized cells or electrical fields (i.e., by limiting the electrical field beyond the target site). Localized cells may be created by, for example, spacing one or more electrodes 44 very close together or biasing the electrical field with conductors and/or magnetic fields. For example, electrical fields may be localized or shaped by using electrodes 44 with different geometries, by using one or more multiple electrodes, and/or by modifying the frequency, pulse-width, voltage, stimulation waveforms, paired pulses, sequential pulses, and/or combinations thereof.

It should also be appreciated that more than one implantable medical device 10 may be used to treat a pulmonary condition. For example, it may be desirable to modulate the SNS of a subject by placing one implantable medical device 10 over an anterior portion of a tracheal surface and another implantable medical device over a portion of the sympathetic trunk 20. Alternatively, it may be desirable to modulate the PNS by placing one implantable medical device 10 over a posterior portion of a tracheal surface and another implantable medical device over a portion of the vagus nerve.

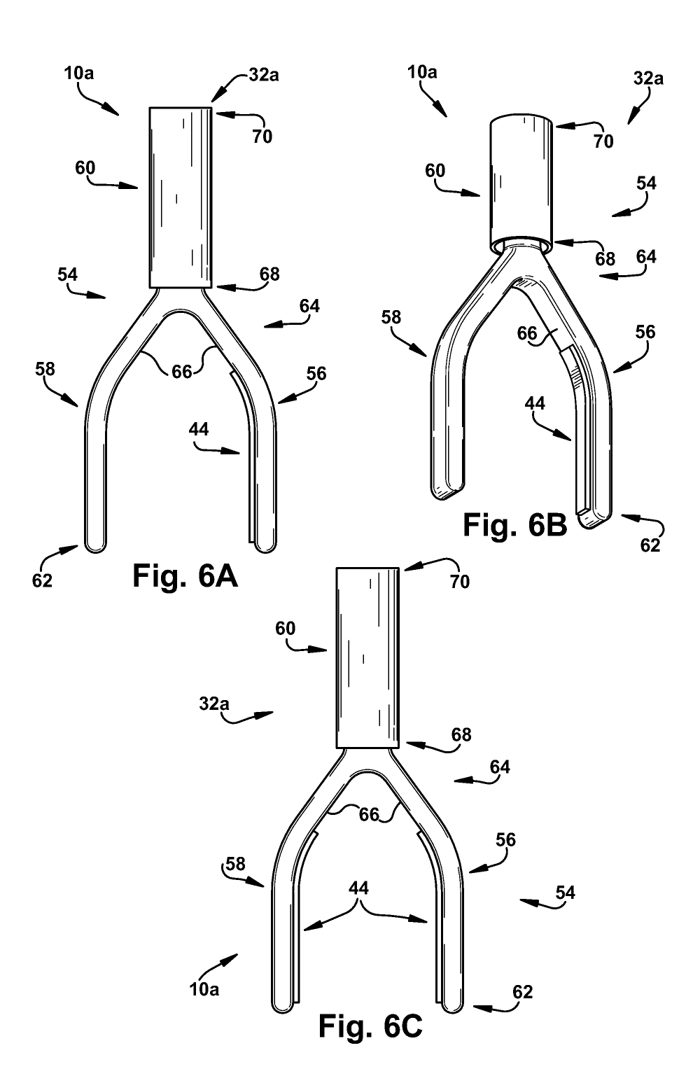

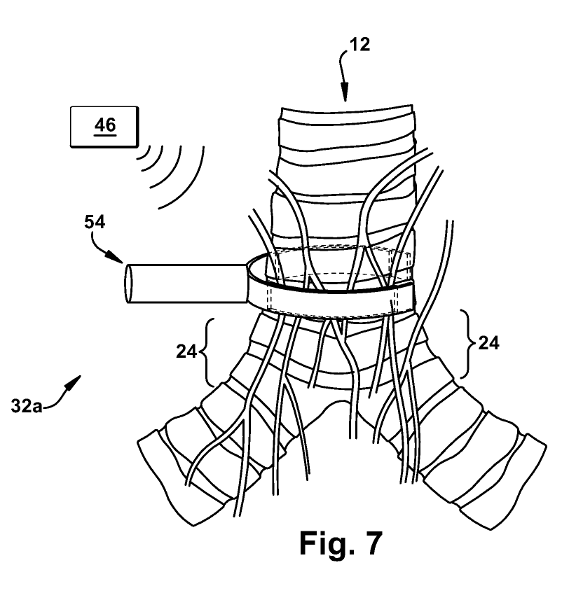

Another embodiment of the present invention is illustrated in FIGS. 6A-7. The implantable medical device 10_{α} shown in FIGS. 6A-7 is identically constructed as the implantable medical device 10 shown in FIGS. 1A and 1B, except as described below. In FIGS. 6A-7, structures that are identical as structures in FIGS. 1A and 1B use the same reference numbers, whereas structures that are similar but not identical carry the suffix “a”.

As shown in FIGS. 6A-C, the implantable medical device 10_{α} can include an implantable electrode assembly 32_{α} for treating a pulmonary condition in a subject. The implantable electrode assembly 32_{α} can comprise a clip member 54 having a generally Y-shaped configuration and being constructed in a manner similar to the hemostatic clips commercially available from Olympus America, Inc. (Center Valley, Pa.). Alternatively, the clip member 54 can be constructed identically or similarly to a binder clip (or banker's clip) (not shown). Other examples of implantable medical devices to which the clip member can be identically or similarly constructed include those disclosed in U.S. Patent Pub. No. 2006/0155344 A1 and U.S. Pat. No. 6,885,888, the entireties of which are hereby incorporated by reference.

The clip member 54 can have first and second flexible arm members 56 and 58 integrally formed with an attachment member 60. As shown in FIG. 6A, the first and second flexible arm members 56 and 58 can have a C-shaped configuration to facilitate placement of the clip member 54 about an extraluminal target site. It will be appreciated that the flexible arm members 56 and 58 can have other configurations, such as U- or V-shaped configurations, and may have axial lengths which are greater or lesser than the axial lengths shown in FIGS. 6A and 6B. The flexible arm members 56 and 58 can be made of a biocompatible, medical grade material, such as DACRON, GORETEX, woven velour, polyurethane, or heparin-coated fabric, graphite, ceramic, hardened plastics, cobalt-nickel alloys (e.g., Elgiloy), titanium, nickel-titanium alloys (e.g., Nitinol), cobalt-chromium alloys (e.g., Stellite), nickel-cobalt-chromium-molybdenum alloys (e.g., MP35N), and stainless steel.

Each of the flexible arm members 56 and 58 can include a first end portion and a second end portion 62 and 64. The first end portion 62 of each of the first and second flexible arm members 56 and 58 can be spaced apart in an open configuration (FIG. 6A) to permit attachment of the clip member 54 to a target site. Additionally, the first end portion 62 of each of the first and second arm members 56 and 58 can be in contact with one another in a closed configuration (not shown) to facilitate implantation of the clip member 54, as well as to facilitate securing of the clip member about a target site.

Each of the flexible arm members 56 and 58 can include an inner surface 66 for receiving an electrode 44 and for contacting a target site. As shown in FIGS. 6A and 6B, for example, the inner surface 66 of the first flexible arm member 56 can include an electrode 44 operably coupled thereto. The electrode 44 can extend across a portion of the inner surface 66 or, alternatively, across the entire inner surface. It will be appreciated that an electrode 44 may also be operably coupled to the inner surface 66 of the second flexible arm member 58 (FIG. 6C).

The second end portion 64 of each of the first and second flexible arm members 56 and 58 can be integrally formed with a first end 68 of the attachment member 60. The attachment member 60 can have a cylindrical shape (or any other shape) to facilitate placement of the clip member 54 at a target site. In particular, a second end 70 of the attachment member 60 can include an attachment mechanism (not shown) to facilitate delivery of the clip member 54 via a delivery device (not shown), such as an endoscope. For example, the attachment mechanism can be identical or similar to the attachment mechanism(s) used to deliver the hemostatic clips commercially available from Olympus America, Inc.

The implantable electrode assembly 32_{α} illustrated in FIGS. 6A-7 can be used to treat a pulmonary condition in a subject, such as asthma. One step of the method can include providing an implantable electrode assembly 32_{α}, such as the one shown in FIGS. 6A and 6B. A direct approach similar or identical to the one described above for the implantable electrode assembly 32 shown in FIGS. 1A and 1B can be used to implant the implantable electrode assembly 32_{α}. For example, the implantable electrode assembly 32_{α} can be implanted at a target site comprising an extraluminal portion of the distal trachea 12 which is innervated by a portion of the anterior pulmonary plexus 24.

Prior to implanting the implantable electrode assembly 32_{α}, the anatomical dimensions of the target site can be determined using known methods, such as MRI or CT. After determining the dimensions of the target site, an appropriately-sized implantable electrode assembly 32_{α} can be selected for implantation. Any one or combination of known surgical approaches can be used to implant the implantable electrode assembly 32_{α}. Examples of suitable approaches include, but are not limited to, trans-tracheal, trans-mediastinal, transvenous (e.g., through the pulmonary trunk), trans-aortic, trans-esophageal routes, trans-thoracic, percutaneous, posterior para-spinal, anterior transcutaneous, subcutaneous, and transmediastinal routes. To facilitate placement of the implantable electrode assembly 32_{α} at the target site, at least a portion of the implantable electrode assembly may be made of a radio-opaque material or include radio-opaque markers (not shown) to facilitate fluoroscopic visualization.

Where a trans-mediastinal approach is used, for example, an anterior mediastinoscopy or thoracoscopy may be used to place the implantable electrode assembly 32_{α}. The advantage of thoracoscopy is the visibility, even to the subcarinal anterior mediastinum. The mediastinoscopy can be performed under general anesthesia using standard KARL STORZ mediastinoscope, either with or without video assistance. An incision can be made in the suprasternal notch and dissection performed caudally to the thyroid isthmus and then continued by blunt dissection into the pretracheal space.

Once the carina 30 has been identified, the implantable electrode assembly 32_{α} can be advanced to the distal-most part of trachea 12 above the surrounding soft tissue. For example, the first and second flexible arm members 56 and 58 can be placed in an open configuration, and the clip member 54 then manipulated so that the flexible arm members envelop a portion of the tracheal wall innervated by the anterior pulmonary plexus 24. The clip member 54 can then be positioned such that the electrode 44 is in direct contact with a portion of the anterior pulmonary plexus 24.

After the implantable electrode assembly 32_{α} has been appropriately positioned at the target site, electric current can be delivered to the implantable electrode assembly. It will be appreciated, however, that the implantable electrode assembly 32_{α} can be positioned at the target site such that a portion of the electrode 44 contacts the external tracheal wall at any portion (e.g., anterior, posterior, right, left) and at any degree or angle (e.g., from about 5 degrees to about 360 degrees). It should be appreciated that an alternative implantation approach could include transillumination of the skin via a bronchoscopy device. Using such an approach, the bronchoscopy device could delineate the target site via a light. The light, in turn, could trans-illuminate the trachea 12 and bronchi in a dorsal direction. Additionally or optionally, the light could further serve as a percutaneous guide when approaching the target site.

As shown in FIG. 7, RF energy can then be delivered to the implantable electrode assembly 32_{α} via a wirelessly-coupled energy delivery source 46. The electric current can then be directed to the electrode 44 via a controller which directs and apportions a desired amount of electric current to the electrode. As electric current is delivered to the electrode 44, the electrode can conduct electric current through the anterior tracheal wall to the anterior pulmonary plexus 24. For example, electrical stimulation or activation of the anterior pulmonary plexus 24 can increase sympathetic nerve function, in turn causing the bronchioles to dilate and thus treat the asthmatic symptoms of the subject.

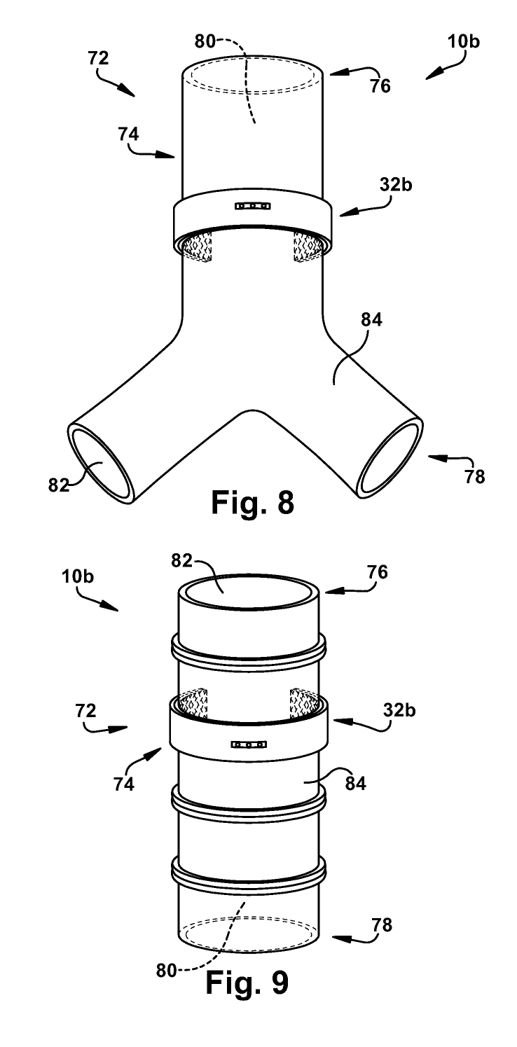

Another embodiment of the present invention is illustrated in FIGS. 8-11. The implantable medical device 10_{b} shown in FIGS. 8-11 is identically constructed as the implantable medical device 10 shown in FIGS. 1A and 1B, except as described below. In FIGS. 8-11, structures that are identical as structures in FIGS. 1A and 1B use the same reference numbers, whereas structures that are similar but not identical carry the suffix “b”.

As shown in FIGS. 8 and 9, the implantable medical device 10_{b} can include an endotracheal apparatus 72 for treating a pulmonary condition. The endotracheal apparatus 72 can comprise a stent member 74 operably coupled to an electrode assembly 32_{b}. The stent member 74 can include first and second ends 76 and 78 and a lumen 80 extending between the ends. The lumen 80 can define an inner surface 82 opposite an outer surface 84. The stent member 74 can have a rigid, semi-rigid, or flexible configuration, and be made of any one or combination of biocompatible, medical grade materials, such as silicone. The stent member 74 can have any variety of shapes and sizes, including a Y-shaped configuration (FIG. 8) or a cylinder-shaped configuration (FIG. 9). Non-limiting examples of stent members 74 can include the NOVATECH DUMON Y stent and the DUMON TF tracheal stent, which are commercially available from Boston Medical Products, Inc. (Westborough, Mass.).

The electrode assembly 32_{b} can be identically or similarly constructed as the implantable electrode assembly 32 shown in FIGS. 1A and 1B. As shown in FIGS. 8 and 9, for example, the electrode 44 can be operably coupled to the second surface 42 of the flexible member 34 such that the first surface 40 of the flexible member is in contact with the outer surface 84 of the stent member 74. The electrode assembly 32_{b} can be coupled to the stent member 74 by friction, an adhesive, clips, staples, sutures, or a combination thereof. As described in more detail below, the position of the electrode assembly 32_{b} about the stent member 74 can be adjusted to optimize the location of the electrode 44 relative to a target site.

In another embodiment of the present invention, a method is provided for treating a pulmonary condition (e.g., asthma) in a subject. One step of the method can include providing an endotracheal apparatus 72, such as the one shown in FIG. 9, for implantation at a target site in the tracheo-bronchial tree 22 of a subject. For example, the target site can include a distal portion of the trachea 12 adjacent the carina 30 which is innervated by the anterior pulmonary plexus 24.

Prior to implantation of the endotracheal apparatus 72, the anatomical dimensions of the target site can be determined using known methods, such as CT, MRI, and/or visual inspection via endoscopy. Next, an appropriately-sized endotracheal apparatus 72 can be selected for implantation. The electrode assembly 32_{b} should be positioned about the stent member 74 such that the orientation of the electrode 44, upon implantation of the endotracheal apparatus 72, is substantially adjacent a portion of the anterior pulmonary plexus 24.

After selecting an appropriately-sized endotracheal apparatus 72, the endotracheal apparatus can be implanted using any one or combination of known surgical approaches. Examples of suitable approaches include, but are not limited to, trans-tracheal, trans-mediastinal, transvenous (e.g., through the pulmonary trunk), trans-aortic, trans-esophageal routes, trans-thoracic, percutaneous, posterior para-spinal, anterior transcutaneous, subcutaneous, and transmediastinal routes. To facilitate placement of the endotracheal apparatus 72 at the target site, at least a portion of the endotracheal apparatus may be made of a radio-opaque material or include radio-opaque markers (not shown) to facilitate fluoroscopic visualization.

Where an endoscopic approach is used, for example, the endotracheal apparatus 72 can be implanted at the target site using a standard bronchoscope, such that the electrode 44 contacts an anterior portion of a distal tracheal wall. After the endotracheal apparatus 72 has been appropriately positioned at the target site, electric current can be delivered to the electrode assembly 32_{b}. As shown in FIG. 11, RF energy can be delivered to the electrode assembly 32_{b} via a wirelessly-coupled energy delivery source 46. The electric current can then be directed to the electrode 44 via a controller which directs and apportions a desired amount of electric current to the electrode. As electric current is delivered to the electrode 44, the electrode conducts electric current through the tracheal wall to the anterior pulmonary plexus 24. For example, electrical stimulation or activation of the anterior pulmonary plexus 24 can increase sympathetic nerve function, in turn causing the bronchioles to dilate and thus treat the asthmatic symptoms of the subject.

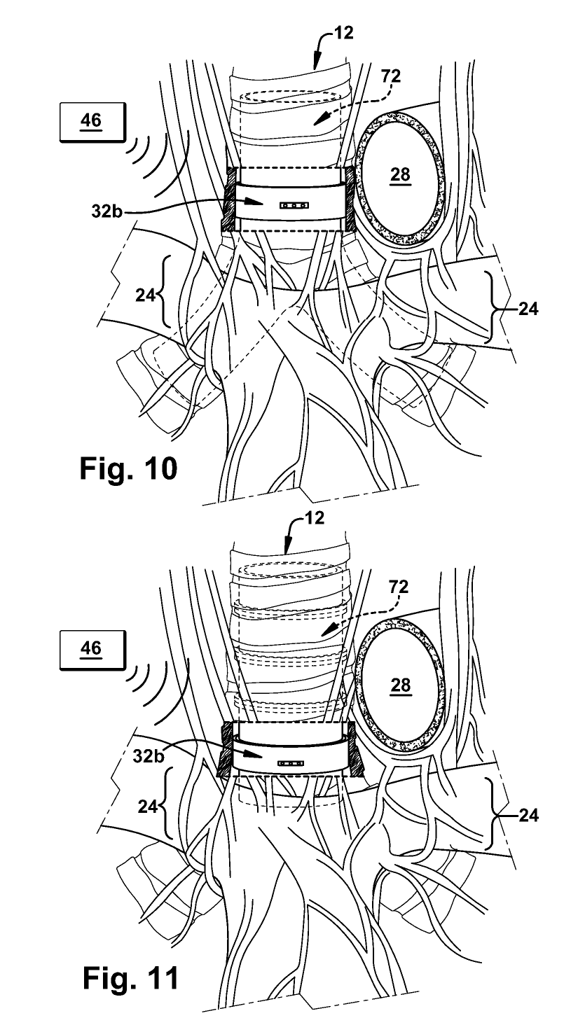

From the above description of the invention, those skilled in the art will perceive improvements, changes and modifications. For example, it will be appreciated that the endotracheal apparatus 72 shown in FIG. 8 can be implanted at a distal portion of the trachea 12 as shown in FIG. 10. Such improvements, changes, and modifications are within the skill of the art and are intended to be covered by the appended claims.

Having described the invention, we claim:

1. A method for treating a pulmonary condition in a subject, said method comprising the steps of:
providing an implantable electrode assembly, the implantable electrode assembly comprising a flexible member, at least one electrode operably secured to the flexible member, and an attachment mechanism for securing the at least one electrode to the flexible member;
implanting the implantable electrode assembly at a target site, the target site comprising an intraluminal location innervated by at least one nerve of the autonomic nervous system (ANS);
positioning the implantable electrode assembly at the target site such that the flexible member conforms to a portion of an inner surface of the target site and the at least one electrode is positioned substantially adjacent the at least one nerve; and
delivering electric current to the at least one electrode to effect a change in the ANS of the subject.

2. The method of claim 1, wherein the intraluminal target site comprises an inner surface of an anatomical structure selected from the group consisting of a blood vessel, the esophagus, the tracheo-bronchial tree, the primary bronchi, and the distal bronchi.

3. The method of claim 2, the intraluminal target site comprising an inner surface of the trachea adjacent the carina.

4. The method of claim 1, wherein the at least one nerve is selected from the group consisting of a spinal nerve, a preganglionic fiber of a spinal nerve, a postganglionic fiber of a spinal nerve, a sympathetic chain ganglion, a thoracic sympathetic chain ganglion, a superior cervical ganglion, a cervical ganglion, a lower cervical ganglion, an inferior cervical ganglion, an intramural ganglion, a splanchnic nerve, an esophageal plexus, a cardiac plexus, a pulmonary plexus, an anterior pulmonary plexus, a posterior pulmonary plexus, a celiac plexus, a hypogastric plexus, an inferior mesenteric ganglion, a celiac ganglion, and a superior mesenteric ganglion.

5. The method of claim 1, wherein delivery of electric current to the at least one electrode effects a change in the parasympathetic nervous system (PNS) of the subject.

6. The method of claim 1, wherein delivery of electric current to the at least one electrode effects a change in the sympathetic nervous system (SNS) of the subject.

7. The method of claim 1, wherein delivery of electric current to the anterior pulmonary plexus effects a change in the SNS.

8. The method of claim 1, wherein delivery of electric current to the posterior pulmonary plexus effects a change in the PNS.

9. The method of claim 1, wherein delivery of electric current to the anterior pulmonary plexus effects a change in both the SNS and the PNS.

10. The method of claim 1, wherein delivery of electric current to the posterior pulmonary plexus effects a change in both the SNS and the PNS.

11. The method of claim 1 further comprising the steps of:
sensing a bodily activity associated with the pulmonary condition;
generating a sensor signal based on the bodily activity; and
activating the implantable electrode assembly to adjust application of electric current to the target site in response to the sensor signal to treat the pulmonary condition.

12. The method of claim 1, wherein the pulmonary condition is selected from the group consisting of genetic conditions, acquired conditions, primary conditions, secondary conditions, asthma, chronic obstructive pulmonary disease, cystic fibrosis, bronchiolitis, pneumonia, bronchitis, emphysema, adult respiratory distress syndrome, allergies, lung cancer, small cell lung cancer, primary lung cancer, metastatic lung cancer, brochiectasis, bronchopulmonary dysplasia, chronic bronchitis, chronic lower respiratory diseases, croup, high altitude pulmonary edema, pulmonary fibrosis, interstitial lung disease, reactive airway disease, lymphangioleiomyomatosis, neonatal respiratory distress syndrome, parainfluenza, pleural effusion, pleurisy, pneumothorax, primary pulmonary hypertension, psittacosis, pulmonary edema secondary to various causes, pulmonary embolism, pulmonary hypertension secondary to various causes, respiratory failure secondary to various causes, sleep apnea, sarcoidosis, smoking, stridor, acute respiratory distress syndrome, infectious diseases, SARS, tuberculosis, psittacosis infection, Q fever, parainfluenza, respiratory syncytial virus, combinations thereof, and conditions caused by any one or combination of the above.

13. An implantable electrode assembly for positioning at a target site and for treating a pulmonary condition in a subject, said implantable electrode assembly comprising:
a flexible member having first and second end portions and oppositely disposed first and second surfaces, said first and second surfaces for securing said flexible member at the target site;
at least one electrode operably coupled to a portion of at least one of said first or second surfaces of said flexible member, said at least one electrode being adapted to selectively deliver electric current to the target site and effect a change in the ANS of the subject; and
an attachment mechanism for securing said at least one electrode to at least one of said first or second surfaces of said flexible member.

14. The implantable electrode assembly of claim 13, wherein said flexible member is adapted to conform to an extraluminal target site.

15. The implantable electrode assembly of claim 13, wherein the extraluminal target site is an outer surface of an anatomical structure selected from the group consisting of a blood vessel, the esophagus, the tracheo-bronchial tree, the primary bronchi, and the distal bronchi.

16. The implantable electrode assembly of claim 15, wherein said flexible member is adapted to conform to an outer surface of the trachea adjacent the carina.

17. The implantable electrode assembly of claim 13, wherein said flexible member is adapted to conform to an intraluminal target site.

18. The implantable electrode assembly of claim 13, wherein the target site is an intraluminal target site comprising an inner surface of an anatomical structure selected from the group consisting of a blood vessel, the esophagus, the tracheo-bronchial tree, the primary bronchi, and the distal bronchi.

19. The implantable electrode assembly of claim 18, wherein said flexible member is adapted to conform to an inner surface of the trachea adjacent the carina.

20. The implantable electrode assembly of claim 13, wherein said flexible member has a C-shaped configuration.

21. The implantable electrode assembly of claim 13, wherein said at least one electrode extends across a portion of said first surface of said flexible member.

22. The implantable electrode assembly of claim 13, wherein said at least one electrode extends across the entire portion of said first surface of said flexible member.

23. The implantable electrode assembly of claim 13, wherein said at least one electrode extends across a portion of said second surface of said flexible member.

24. The implantable electrode assembly of claim 13, wherein said at least one electrode extends across the entire portion of said second surface of said flexible member.

25. The implantable electrode assembly of claim 13, wherein electric current is delivered to said at least one electrode via a wireless energy source.

26. The implantable electrode assembly of claim 13, wherein said attachment mechanism comprises a metal member and a plurality of securing members, said metal member being operably coupled to at least one of said first or second surfaces of said flexible member via said securing members.

27. The implantable electrode assembly of claim 13, wherein at least a portion of said implantable electrode assembly is treated with a therapeutic agent for eluting into vascular tissue, the blood stream, or a combination thereof.

28. The implantable electrode assembly of claim 27, wherein a plurality of portions of said implantable electrode assembly are separately treated with a different one of said at least one therapeutic agent.

29. The implantable electrode assembly of claim 13, wherein at least a portion of said implantable electrode assembly is covered with a layer of biocompatible material.

30. The implantable electrode assembly of claim 13, wherein a sensor capable of sensing a bodily activity associated with the pulmonary condition is associated with said implantable electrode assembly.

31. The implantable electrode assembly of claim 30, wherein said sensor is operably coupled to a portion of said implantable electrode assembly.

32. The implantable electrode assembly of claim 13, the pulmonary disorder being selected from the group consisting of genetic conditions, acquired conditions, primary conditions, secondary conditions, asthma, chronic obstructive pulmonary disease, cystic fibrosis, bronchiolitis, pneumonia, bronchitis, emphysema, adult respiratory distress syndrome, allergies, lung cancer, small cell lung cancer, primary lung cancer, metastatic lung cancer, brochiectasis, bronchopulmonary dysplasia, chronic bronchitis, chronic lower respiratory diseases, croup, high altitude pulmonary edema, pulmonary fibrosis, interstitial lung disease, reactive airway disease, lymphangioleiomyomatosis, neonatal respiratory distress syndrome, parainfluenza, pleural effusion, pleurisy, pneumothorax, primary pulmonary hypertension, psittacosis, pulmonary edema secondary to various causes, pulmonary embolism, pulmonary hypertension secondary to various causes, respiratory failure secondary to various causes, sleep apnea, sarcoidosis, smoking, stridor, acute respiratory distress syndrome, infectious diseases, SARS, tuberculosis, psittacosis infection, Q fever, parainfluenza, respiratory syncytial virus, combinations thereof, and conditions caused by any one or combination of the above.

33. The implantable electrode assembly of claim 13, wherein the at least one nerve is selected from the group consisting of a spinal nerve, a preganglionic fiber of a spinal nerve, a postganglionic fiber of a spinal nerve, a sympathetic chain ganglion, a thoracic sympathetic chain ganglion, a cervical ganglion, a superior cervical ganglion, a lower cervical ganglion, an inferior cervical ganglion, an intramural ganglion, a splanchnic nerve, an esophageal plexus, a cardiac plexus, a pulmonary plexus, an anterior pulmonary plexus, a posterior pulmonary plexus, a celiac plexus, a hypogastric plexus, an inferior mesenteric ganglion, a celiac ganglion, and a superior mesenteric ganglion.

34. A method for treating a pulmonary condition in a subject, said method comprising the steps of:
providing an implantable electrode assembly, the implantable electrode assembly comprising a flexible member, at least one electrode operably secured to the flexible member, and an attachment mechanism for securing the at least one electrode to the flexible member;
implanting the implantable electrode assembly at a target site, the target site comprising an extraluminal location innervated by at least one nerve of the ANS;
positioning the implantable electrode assembly at the target site such that the flexible member envelops a portion of the target site and the at least one electrode is in direct contact with the at one least nerve; and
delivering electric current to the at least one electrode to effect a change in the ANS of the subject.

35. The method of claim 34, wherein the extraluminal target site comprises an outer surface of an anatomical structure selected from the group consisting of a blood vessel, the esophagus, the tracheo-bronchial tree, the primary bronchi, and the distal bronchi.

36. The method of claim 34, wherein the extraluminal target site comprises an outer surface of the trachea adjacent the carina.

37. The method of claim 34, wherein the at least one nerve is selected from the group consisting of a spinal nerve, a preganglionic fiber of a spinal nerve, a postganglionic fiber of a spinal nerve, a sympathetic chain ganglion, a thoracic sympathetic chain ganglion, a cervical ganglion, a superior cervical ganglion, a lower cervical ganglion, an inferior cervical ganglion, an intramural ganglion, a splanchnic nerve, an esophageal plexus, a cardiac plexus, a pulmonary plexus, an anterior pulmonary plexus, a posterior pulmonary plexus, a celiac plexus, a hypogastric plexus, an inferior mesenteric ganglion, a celiac ganglion, and a superior mesenteric ganglion.

39. The method of claim 34, wherein delivery of electric current to the at least one electrode effects a change in the PNS of the subject.

40. The method of claim 34, wherein delivery of electric current to the at least one electrode effects a change in the SNS of the subject.

41. The method of claim 34, wherein delivery of electric current to the anterior pulmonary plexus effects a change in the SNS.

42. The method of claim 34, wherein delivery of electric current to the posterior pulmonary plexus effects a change in the PNS.

43. The method of claim 34, wherein delivery of electric current to the anterior pulmonary plexus effects a change in both the SNS and the PNS.

44. The method of claim 34, wherein delivery of electric current to the posterior pulmonary plexus effects a change in both the SNS and the PNS.

45. The method of claim 34 further comprising the steps of:
sensing a bodily activity associated with the pulmonary condition;
generating a sensor signal based on the bodily activity; and
activating the implantable electrode assembly to adjust application of electric current to the target site in response to the sensor signal to treat the pulmonary condition.

46. The method of claim 34, wherein the pulmonary condition is selected from the group consisting of genetic conditions, acquired conditions, primary conditions, secondary conditions, asthma, chronic obstructive pulmonary disease, cystic fibrosis, bronchiolitis, pneumonia, bronchitis, emphysema, adult respiratory distress syndrome, allergies, lung cancer, small cell lung cancer, primary lung cancer, metastatic lung cancer, brochiectasis, bronchopulmonary dysplasia, chronic bronchitis, chronic lower respiratory diseases, croup, high altitude pulmonary edema, pulmonary fibrosis, interstitial lung disease, reactive airway disease, lymphangioleiomyomatosis, neonatal respiratory distress syndrome, parainfluenza, pleural effusion, pleurisy, pneumothorax, primary pulmonary hypertension, psittacosis, pulmonary edema secondary to various causes, pulmonary embolism, pulmonary hypertension secondary to various causes, respiratory failure secondary to various causes, sleep apnea, sarcoidosis, smoking, stridor, acute respiratory distress syndrome, infectious diseases, SARS, tuberculosis, psittacosis infection, Q fever, parainfluenza, respiratory syncytial virus, combinations thereof, and conditions caused by any one or combination of the above.

47. An implantable electrode assembly for treating a pulmonary condition in a subject, said implantable electrode assembly comprising:
a clip member including first and second flexible arm members integrally formed with an attachment member, said flexible arm members being adapted for placement about a target site, the target site comprising an extraluminal location innervated by at least one nerve of the ANS; and
an electrode operably coupled to at least one of said first or second flexible arm members, said electrode being adapted to selectively deliver electric current to the target site and effect a change in the ANS of the subject.

48. The implantable electrode assembly of claim 47, wherein said first and second flexible arm members respectively include first and second electrodes operably coupled thereto.

49. The implantable electrode assembly of claim 47, wherein the target site comprises an outer surface of an anatomical structure selected from the group consisting of a blood vessel, the esophagus, the tracheo-bronchial tree, the primary bronchi, and the distal bronchi.

50. The implantable electrode assembly of claim 49, wherein the target site comprises an outer surface of the trachea adjacent the carina.

51. The implantable electrode assembly of claim 47, wherein electric current is delivered to the electrode via a wireless energy source.

52. The implantable electrode assembly of claim 47, wherein the at least one nerve is selected from the group consisting of a spinal nerve, a preganglionic fiber of a spinal nerve, a postganglionic fiber of a spinal nerve, a sympathetic chain ganglion, a thoracic sympathetic chain ganglion, a cervical ganglion, a superior cervical ganglion, a lower cervical ganglion, an inferior cervical ganglion, an intramural ganglion, a splanchnic nerve, an esophageal plexus, a cardiac plexus, a pulmonary plexus, an anterior pulmonary plexus, a posterior pulmonary plexus, a celiac plexus, a hypogastric plexus, an inferior mesenteric ganglion, a celiac ganglion, and a superior mesenteric ganglion.

53. The implantable electrode assembly of claim 47, wherein the pulmonary condition is selected from the group consisting of genetic conditions, acquired conditions, primary conditions, secondary conditions, asthma, chronic obstructive pulmonary disease, cystic fibrosis, bronchiolitis, pneumonia, bronchitis, emphysema, adult respiratory distress syndrome, allergies, lung cancer, small cell lung cancer, primary lung cancer, metastatic lung cancer, brochiectasis, bronchopulmonary dysplasia, chronic bronchitis, chronic lower respiratory diseases, croup, high altitude pulmonary edema, pulmonary fibrosis, interstitial lung disease, reactive airway disease, lymphangioleiomyomatosis, neonatal respiratory distress syndrome, parainfluenza, pleural effusion, pleurisy, pneumothorax, primary pulmonary hypertension, psittacosis, pulmonary edema secondary to various causes, pulmonary embolism, pulmonary hypertension secondary to various causes, respiratory failure secondary to various causes, sleep apnea, sarcoidosis, smoking, stridor, acute respiratory distress syndrome, infectious diseases, SARS, tuberculosis, psittacosis infection, Q fever, parainfluenza, respiratory syncytial virus, combinations thereof, and conditions caused by any one or combination of the above.

54. The implantable electrode assembly of claim 47, wherein at least a portion of said implantable electrode assembly is treated with a therapeutic agent for eluting into vascular tissue, the blood stream, or a combination thereof.

55. The implantable electrode assembly of claim 54, wherein a plurality of portions of said implantable electrode assembly are separately treated with a different one of said at least one therapeutic agent.

56. The implantable electrode assembly of claim 47, wherein at least a portion of said implantable electrode assembly is covered with a layer of biocompatible material.

57. The implantable electrode assembly of claim 47, wherein a sensor capable of sensing a bodily activity associated with the pulmonary condition is associated with said implantable electrode assembly.

58. The implantable electrode assembly of claim 57, wherein said sensor is operably coupled to a portion of said implantable electrode assembly.

59. A method for treating a pulmonary condition in a subject, said method comprising the steps of:
providing an implantable electrode assembly, the implantable electrode assembly comprising a clip member and an electrode, the clip member including first and second flexible arm members integrally formed with an attachment member, the flexible arm members being adapted for placement about a target site comprising an extraluminal location innervated by at least one nerve of the ANS, the electrode being operably coupled to at least one of the first or second flexible arm members and being adapted to selectively deliver electric current to the target site and effect a change in the ANS of the subject;
implanting the implantable electrode assembly at the target site;
positioning the implantable electrode assembly such that the first and second flexible arm members envelop a portion of the target site and the electrode is in direct contact with the at least one nerve; and
delivering electric current to the electrode to effect a change in the ANS of the subject.

60. The method of claim 59 further comprising the steps of:
attaching the attachment member to a delivery apparatus;
inserting the implantable electrode assembly into the subject; and
placing the first and second arm members adjacent the target site.

61. The method of claim 59, wherein the at least one nerve is selected from the group consisting of a spinal nerve, a preganglionic fiber of a spinal nerve, a postganglionic fiber of a spinal nerve, a sympathetic chain ganglion, a thoracic sympathetic chain ganglion, a cervical ganglion, a superior cervical ganglion, a lower cervical ganglion, an inferior cervical ganglion, an intramural ganglion, a splanchnic nerve, an esophageal plexus, a cardiac plexus, a pulmonary plexus, an anterior pulmonary plexus, a posterior pulmonary plexus, a celiac plexus, a hypogastric plexus, an inferior mesenteric ganglion, a celiac ganglion, and a superior mesenteric ganglion.

62. The method of claim 59, wherein delivery of electric current effects a change in the PNS of the subject.

63. The method of claim 59, wherein delivery of electric current effects a change in the ANS of the subject.

64 The method of claim 59, wherein electric current is delivered to the anterior pulmonary plexus to effect a change in the SNS.

65. The method of claim 59, wherein electric current is delivered to the posterior pulmonary plexus to effect a change in the PNS.

66. The method of claim 59, wherein delivery of electric current to the anterior pulmonary plexus effects a change in both the SNS and the PNS.

67. The method of claim 59, wherein delivery of electric current to the posterior pulmonary plexus effects a change in both the SNS and the PNS.

68. The method of claim 59, wherein the pulmonary condition is selected from the group consisting of genetic conditions, acquired conditions, primary conditions, secondary conditions, asthma, chronic obstructive pulmonary disease, cystic fibrosis, bronchiolitis, pneumonia, bronchitis, emphysema, adult respiratory distress syndrome, allergies, lung cancer, small cell lung cancer, primary lung cancer, metastatic lung cancer, brochiectasis, bronchopulmonary dysplasia, chronic bronchitis, chronic lower respiratory diseases, croup, high altitude pulmonary edema, pulmonary fibrosis, interstitial lung disease, reactive airway disease, lymphangioleiomyomatosis, neonatal respiratory distress syndrome, parainfluenza, pleural effusion, pleurisy, pneumothorax, primary pulmonary hypertension, psittacosis, pulmonary edema secondary to various causes, pulmonary embolism, pulmonary hypertension secondary to various causes, respiratory failure secondary to various causes, sleep apnea, sarcoidosis, smoking, stridor, acute respiratory distress syndrome, infectious diseases, SARS, tuberculosis, psittacosis infection, Q fever, parainfluenza, respiratory syncytial virus, combinations thereof, and conditions caused by any one or combination of the above.
